(19) United States
(12) Patent Application Publication
(10) Pub. No.: US 2020/0395101 A1
(43) Pub. Date: Dec. 17, 2020

(54) POLYMER MOLECULE MODEL DISPOSITION METHOD, SIMULATION METHOD, SIMULATION APPARATUS, AND NON-TRANSITORY COMPUTER-READABLE MEDIUM STORING PROGRAM

(71) Applicant: SUMITOMO HEAVY INDUSTRIES, LTD., Tokyo (JP)

(72) Inventor: Ryota Hirose, Kanagawa (JP)

(21) Appl. No.: 16/783,788

(22) Filed: Feb. 6, 2020

(30) Foreign Application Priority Data
Jun. 12, 2019 (JP) 2019-109712

(51) Int. Cl. G16C 20/10; G06F 30/25; G06K 9/62; G16C 10/00; G06F 17/16

(52) U.S. Cl. CPC G16C 20/10; G06F 30/25; G06F 17/16; G16C 10/00; G06K 9/6215

(57) ABSTRACT

In disposing plural polymer molecule models each including plural monomer particles in an initial disposition area in a virtual space as a simulation target, one monomer particle is disposed in the initial disposition area. A candidate of a position of a monomer particle to be disposed next is determined as a candidate position. When a distance from a midpoint of a line connecting a position of a monomer particle disposed immediately before to a position of a monomer particle to be disposed next to a midpoint of a line connecting positions of two consecutive monomer particles included in a polymer molecule model already disposed does not satisfy an allowable condition, the candidate position determined immediately before is canceled, and another position is determined as a new candidate position. When the distance between the midpoints satisfies the allowable condition, the monomer particle is disposed at the candidate position determined immediately before.

### RELATED APPLICATIONS

The content of Japanese Patent Application No. 2019-109712, on the basis of which priority benefits are claimed in an accompanying application data sheet, is in its entirety incorporated herein by reference.

### BACKGROUND

### Technical Field

Certain embodiments of the present invention relate to a polymer molecule model disposition method, a simulation method, a simulation apparatus, and a non-transitory computer-readable medium storing a program.

### Description of Related Art

There is known a method for simulating a behavior of a polymer material using a molecular dynamics method or a renormalization group molecular dynamics method (in this specification, simply referred to as a “molecular dynamics method”). According to a related art method, a plurality of polymer molecule models (corresponding to polymer molecules) each of which includes a plurality of polymer particle models (corresponding to monomers) are disposed in an initial disposition area in a virtual space. Thereafter, a stable disposition of a plurality of polymer molecule models is determined by performing relaxation calculation based on the molecular dynamics method. Then, behaviors of the plurality of polymer molecule models are analyzed using the molecular dynamics method.

### SUMMARY

According to an embodiment of the present invention, there is provided a polymer molecule model disposition method for disposing a plurality of polymer molecule models each including a plurality of monomer particles in an initial disposition area in a virtual space that is a simulation target, including:

disposing one monomer particle in the initial disposition area;
determining a candidate of a position of a monomer particle to be disposed next as a candidate position; and
canceling, in a case where a distance between midpoints that is a distance from a midpoint of a line connecting a position of a monomer particle that is disposed immediately before to the candidate position of the monomer particle to be disposed next to a midpoint of a line connecting positions of two monomer particles included in a polymer molecule model that is already disposed does not satisfy an allowable condition, the candidate position that is determined immediately before, determining another position as a new candidate position, and disposing, in a case where the distance between midpoints satisfies the allowable condition, the monomer particle at the candidate position that is determined immediately before.

According to another aspect of the invention, there is provided a simulation method including:

disposing a plurality of polymers in a virtual space that is a simulation target using the above-described polymer molecule model disposition method; and
analyzing a behavior of a plurality of polymer molecule models using a molecular dynamics method.

According to still another aspect of the invention, there is provided a simulation apparatus including:

an input unit through which information for defining an initial disposition area of a polymer molecule model that is a simulation target is input; and
a processing unit that disposes a plurality of polymer molecule models each including a plurality of monomer particles in the initial disposition area defined by the information input through the input unit,
in which the processing unit includes
a function of disposing one monomer particle in the initial disposition area, a function of determining a candidate of a position of a monomer particle to be disposed next as a candidate position, and
a function of canceling, in a case where a distance between midpoints that is a distance from a midpoint of a line connecting a position of a monomer particle that is disposed immediately before to the position of the monomer particle to be disposed next to a midpoint of a line connecting positions of two consecutive monomer particles included in a polymer molecule model that is already disposed does not satisfy an allowable condition, the candidate position that is determined immediately before, and determining another position as a new candidate position.

According to still another aspect of the invention, there is a non-transitory computer-readable medium storing a program causing a computer to execute:

a function of acquiring information for defining an initial disposition area where a polymer molecule model that is a simulation target is disposed;
a function of disposing one monomer particle in the initial disposition area in disposing a plurality of polymer molecule models each including a plurality of monomer particles; a function of determining a candidate of a position of a monomer particle to be disposed next as a candidate position; and
a function of canceling, in a case where a distance between midpoints that is a distance from a midpoint of a line connecting a position of a monomer particle that is disposed immediately before to the position of the monomer particle to be disposed next to a midpoint of a line connecting positions of two consecutive monomer particles included in a polymer molecule model that is already disposed does not satisfy an allowable condition, the candidate position that is determined immediately before, and determining another position as a new candidate position.

### BRIEF DESCRIPTION OF THE DRAWINGS

FIG. 1A is a schematic diagram showing a process of disposing a plurality of monomer particles of a polymer molecule model in an initial disposition area of a polymer molecule model that is a simulation target, and FIG. 1B is a schematic diagram showing an example of an disposition of monomer particles after a plurality of polymer molecule models are disposed, and FIG. 1C is a schematic diagram showing only monomer particles having a potential energy higher than a certain potential energy after relaxation calculation.

FIG. 2 is a block diagram showing a simulation apparatus according to an embodiment.

FIG. 3 is a schematic diagram showing a polymer material stirrer as an example of a simulation target.

FIG. 4 is a flowchart of a process of disposing polymer molecule models.

FIGS. 5A and 5B are diagrams schematically showing candidate positions of monomer particles that are already disposed, monomer particles that are disposed immediately before, and monomer particles that are to be disposed next.

FIG. 6 is a flowchart of relaxation calculation and a simulation.

FIG. 7A is a graph showing shapes of a general-purpose interaction potential ϕ₁ and a modified general-purpose interaction potential ϕₘ₁, and FIG. 7B is a graph showing shapes of an intra-polymer interaction potential ϕ₂ and a modified intra-polymer interaction potential ϕₘ₂.

### DETAILED DESCRIPTION

The related art discloses a method for obtaining an initial disposition by dividing an initial disposition area of a polymer molecule model into a plurality of voxels using a known related art technique and disposing one monomer particle that forms the polymer molecule model in one voxel. In this method, for example, a serial number is assigned to the monomer particle disposed in the voxel in accordance with a serial number of the voxel. One polymer molecule model (corresponding to one polymer) is formed by a plurality of monomer particles having consecutive serial numbers. There is a case where a distance between two consecutive serial number voxels is extremely long depending on a voxel division algorithm. For example, in a case where serial numbers are sequentially assigned to voxels disposed in one row, the distance between two consecutive serial number voxels becomes extremely long at a location where the row of voxels changes. Further, in a case where a cavity portion such as a hole exists in the initial disposition area where the polymer molecule model is disposed, the distance between two voxels disposed over the cavity portion becomes extremely long.

In a case where two voxels disposed at a long distance are assigned consecutive serial numbers and monomer particles corresponding to the two voxels are included in one polymer molecule model, the polymer molecule model enters in a very unstable state. That is, the monomer particles are in a state having a very large potential energy. Accordingly, it takes a long time for the relaxation calculation up to reaching a stable state.

Further, since the polymer molecule models are disposed in a column direction of voxels, even in a case where the stable state is reached by the relaxation calculation, the influence of the initial state in which the polymer molecule models are disposed in the column direction remains.

Ina case where the polymer molecule models are disposed by the method disclosed in the related art, it is possible to prevent the distance between the monomer particles from becoming extremely long. However, according to an evaluation experiment performed by the inventors of the present application, it was found that monomer particles having an excessively high energy state were generated, and this state was not solved even by performing the relaxation calculation.

It is desirable to provide a polymer molecule model disposition method, a simulation method, a simulation apparatus, and a non-transitory computer-readable medium storing a program capable of suppressing occurrence of monomer particles having an excessively high energy.

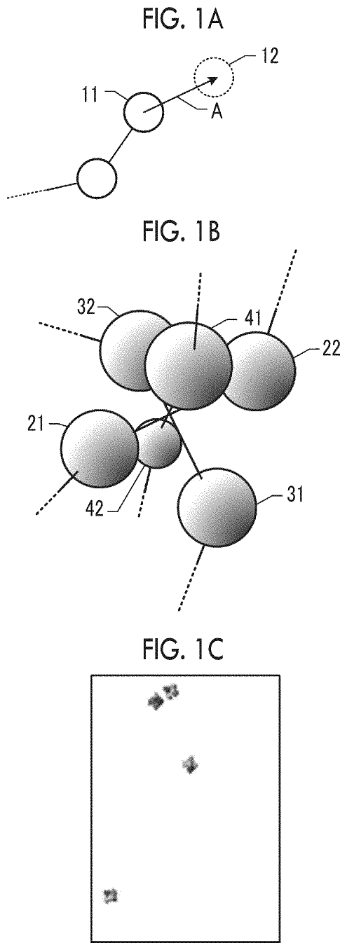

Before describing embodiments of the present invention, a polymer molecule model disposition method according to a reference example will be described with reference to FIGS. 1A to 1C.

FIG. 1A is a schematic diagram showing a process of disposing a plurality of monomer particles of a polymer molecule model in an initial disposition area of a polymer molecule model that is a simulation target. A vector A from a monomer particle 11 that is disposed immediately before toward a monomer particle 12 that is to be disposed next is determined. The direction of the vector A is random, and its length is set in advance. Next, the vector A starting from the position of the monomer particle 12 that is newly disposed is newly determined, and the same operation is repeated. One polymer molecule model is formed by a predetermined number of monomer particles that are disposed in order. In this specification, the “position of the monomer particle” means a center position of the monomer particle.

FIG. 1B is a schematic diagram showing an example of an disposition of monomer particles after a plurality of polymer molecule models are disposed. Two monomer particles 21 and 22 included in one polymer molecule model, two monomer particles 31 and 32 included in another polymer molecule model, and two monomer particles 41 and 42 included in still another polymer molecule model are disposed. For example, a line connecting two monomer particles 21 and 22 and a line connecting the other two monomer particles 31 and 32 cross each other. Aline connecting monomer particles 41 and 42 passes through an area surrounded by the four monomer particles 21, 22, 31, and 32.

Between two monomer particles included in the same polymer molecule model, a finite elongation nonlinear spring potential (FENE potential) acts in addition to an interaction potential that acts between normal particles. Due to the effect of the FENE potential, a distance between two consecutive monomer particles in the same polymer molecule model does not increase up to a certain upper limit or greater. Accordingly, in a case where an disposition state of the six monomer particles 21, 22, 31, 32, 41, and 42 appears as shown in FIG. 1B, this state does not disappear even in a case where relaxation calculation is performed. That is, the three polymer molecule models are entangled and cannot be separated. In this specification, such a state is referred to as a “locked state”. Such a locked state occurs stochastically, and particularly, in a case where the polymer molecule models are disposed at high density, the occurrence probability becomes high.

FIG. 1C is a schematic diagram showing an disposition of monomer particles after relaxation calculation. In FIG. 1C, only monomer particles having a potential energy higher than a certain potential energy are shown. In a case where the relaxation calculation is performed, the monomer particles are almost stationary at a position of a minimum value of an interaction potential and enter a low energy state. However, in the example shown in FIG. 1C, it can be understood that there are monomer particles for which the high potential energy state are not eliminated even after the relaxation calculation. Six monomer particles having a high potential energy appear as a set, and thus, it is considered that a locked state has occurred.

Three polymer molecule models including the locked monomer particles behave as one polymer molecule model having a substantially high degree of polymerization. Accordingly, in a case where a simulation is performed by determining an interaction potential based on actual physical property values such as viscosity, the analysis accuracy is reduced due to a substantial change in the degree of polymerization. Further, since six monomer particles maintaining the locked state have a high potential energy, a time step width should be shortened in order to avoid calculation failure in relaxation calculation or simulation calculation.

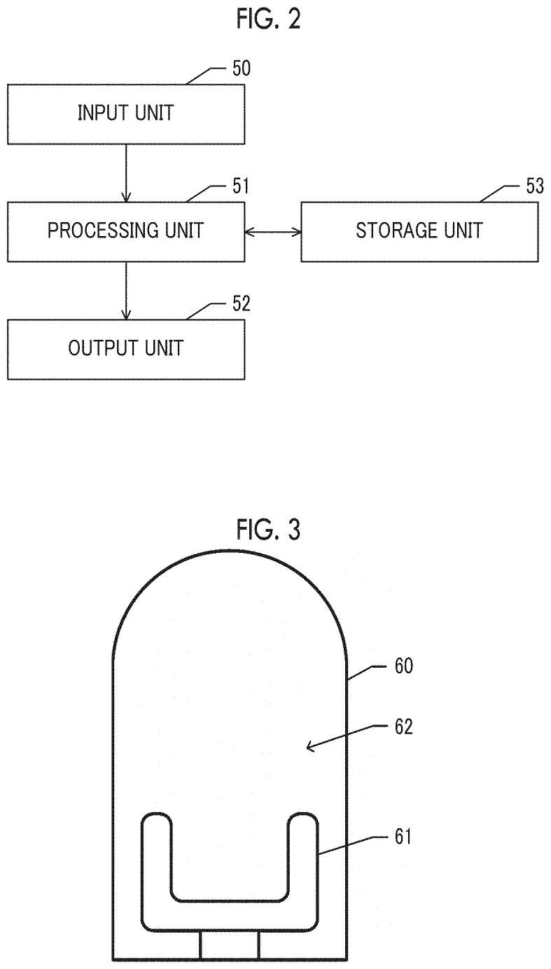

Next, a polymer molecule model disposition method, a simulation method, a simulation apparatus, and a program according to an embodiment will be described with reference to FIGS. 2 to 7B. In the embodiment described below, the appearance of monomer particles in a locked state may be suppressed.

FIG. 2 is a block diagram showing the simulation apparatus according to the embodiment. The simulation apparatus according to the embodiment includes an input unit 50, a processing unit 51, an output unit 52, and a storage unit 53. Simulation conditions are input from the input unit 50. The simulation conditions include, for example, information for defining a three-dimensional shape of a virtual space that is a simulation target, information for defining a three-dimensional shape of an initial disposition area in which a polymer molecule model is disposed, parameters for defining an interaction potential between unspecified monomer particles (hereinafter, a general-purpose interaction potential), parameters for defining an interaction potential between two consecutive monomer particles in the same polymer molecule model (hereinafter, referred to as an intra-polymer interaction potential), the number of monomer particles that form a polymer molecule model (the degree of polymerization), the density of polymer, and the like. Further, various commands or the like are input through the input unit 50 from an operator. The input unit 50 includes, for example, a communication device, a removable medium reading device, a keyboard, a pointing device, and the like.

The processing unit 51 disposes a polymer molecule model on the basis of input conditions, performs a simulation, and outputs a processing result to the output unit 52. The processing result includes position information of monomer particles that forma polymer molecule model, information on energy of the monomer particles, and the like. The processing unit 51 includes, for example, a computer, and executes a program stored in the storage unit 53. The program causes a computer to execute a function of disposing a polymer molecule model, a function of executing a simulation, and the like. The output unit 52 includes a communication device, a removable medium writing device, a display, and the like.

FIG. 3 is a schematic diagram showing a polymer material stirrer as an example of a simulation target. Rotary vanes 61 are contained in a container 60. A polymer material is loaded in the container 60, and as the rotary vanes 61 are rotated, the polymer material is stirred. A space in the container 60 corresponds to a virtual space that is a simulation target. In a case where a polymer molecule model that is a polymer material is initially disposed in the virtual space, the rotary vanes 61 are stationary. An area except for the rotary vanes 61 in the virtual space corresponds to an initial disposition area 62 in which a polymer molecule model is to be disposed before simulation.

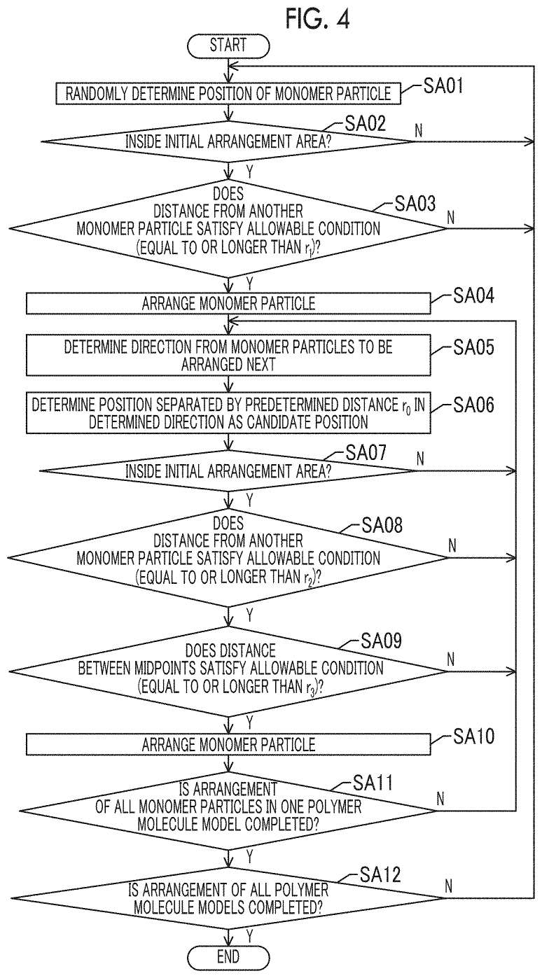

FIG. 4 is a flowchart of a process of disposing a polymer molecule model. Each process shown in FIG. 4 is executed by the processing unit 51 (FIG. 2).

First, the position of one monomer particle is randomly determined in a virtual space that is a simulation target (hereinafter simply referred to as a “virtual space”) (step SA01). It is determined whether or not the position of the monomer particle is within the initial disposition area 62 (FIG. 3) of the polymer molecule model (step SA02), and in a case where the position is outside the initial disposition area 62, the position of the monomer particle is determined again (step SA01). Ina case where the position of the monomer particle is inside the initial disposition area, whether a distance from the newly determined position of the monomer particle to the position of another monomer particle that is already disposed satisfies an allowable condition of an inter-particle distance is determined (step SA03). For example, in a case where the distance from the newly determined position of the monomer particle to the position of another monomer particle that is already disposed is less than a distance r₁, the allowable condition is not satisfied, and in a case where the distance is equal to or longer than the distance r₁, the allowable condition is satisfied. The distance r₁ is determined from the most stable distance derived from the general-purpose interaction potential and the density of the polymer.

In a case where the distance from the newly determined position of the monomer particle to the position of another monomer particle that is already disposed does not satisfy the allowable condition, the position of the monomer particle is determined again (step SA01). In a case where the distance from the newly determined position of the monomer particle to the position of another monomer particle that is already disposed satisfies the allowable condition, a new monomer particle is disposed at the position determined in step SA01 (step SA04).

A direction from a monomer particle that is disposed immediately before to a monomer particle to be disposed next is determined (step SA05). For example, the direction is randomly determined. A position separated by a predetermined distance r₀ from the monomer particle that is disposed immediately before in the direction determined in step SA05 is determined as a candidate position (step SA06). As the distance r₀, for example, the most stable distance derived from a general-purpose interaction potential is adopted. As the distance r₀, the most stable distance derived from a combined potential of the general-purpose interaction potential and the intra-polymer interaction potential may be adopted.

Then, it is determined whether or not the candidate position is inside the initial disposition area 62 (FIG. 3) of the polymer molecule model (step SA07). In a case where the candidate position is outside the initial disposition area 62, a current candidate position is canceled, and a new candidate position is determined (steps SA05 and SA06). In a case where the candidate position is inside the initial disposition area 62, it is determined whether or not a distance from the candidate position to a position of another monomer particle that is already disposed satisfies the allowable condition of the inter-particle distance (step SA08). For example, in a case where the distance from the candidate position to the position of another monomer particle that is already disposed is less than a distance r2, the allowable condition is not satisfied, and in a case where the distance is equal to or longer than the distance r2, the allowable condition is satisfied. The distance r2 is, for example, the same as the distance r1 used as the determination condition in step SA03.

In a case where the candidate position to the position of another monomer particle that is already disposed does not satisfy the allowable condition, the current candidate position is canceled, and a new candidate position is determined (steps SA05 and SA06). In a case where the distance from the candidate position to the position of another monomer particle that is already disposed satisfies the allowable condition, it is determined whether or not a distance LM between midpoints satisfies an allowable condition (step SA09).

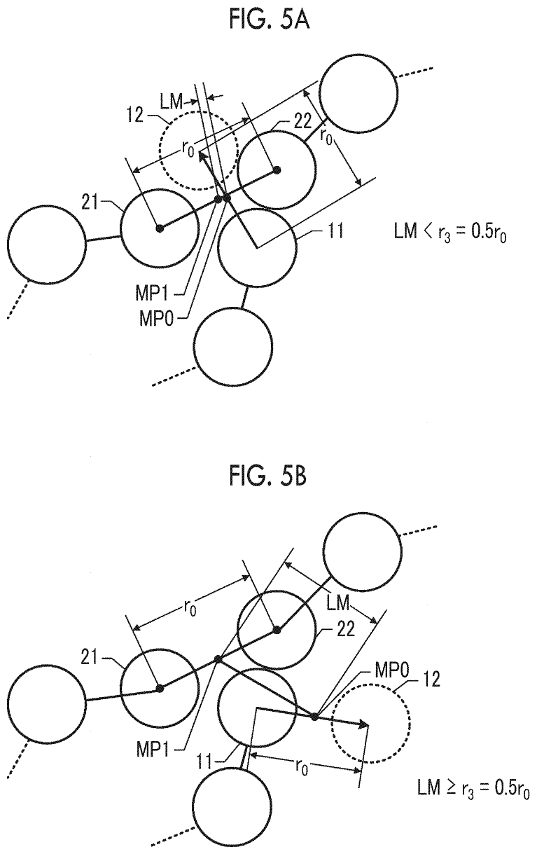

Next, a method for determining whether or not the distance LM between the midpoints satisfies the allowable condition will be described with reference to FIGS. 5A and 5B. FIGS. 5A and 5B are diagrams schematically showing candidate positions of monomer particles 21 and 22 that are already disposed, a monomer particle 11 that is disposed immediately before, and a monomer particle 12 to be disposed next.

A midpoint of a line connecting centers of the two monomer particles 21 and 22 that are already disposed is represented as an MP1. A midpoint of a line connecting a position of the monomer particle 11 that is disposed immediately before and a candidate position of the monomer particle 12 to be disposed next is represented as an MP0. A distance between the midpoint MP1 and the midpoint MP0 is defined as a distance LM between the midpoints. In a case where the distance LM between the midpoints is less than a distance r₃, the distance LM between the midpoints does not satisfy the allowable condition, and in a case where the distance LM is equal to or longer than the distance r₃, the distance LM between the midpoints satisfies the allowable condition. Here, a distance shorter than the above-described distance r₀, for example, 0.5 times the distance r₀ is adopted as the distance r₃, for example. The distance r₀ corresponds to the length of the line connecting the monomer particles 21 and 22 and the length of the line connecting the candidate positions of the monomer particle 11 and of the monomer particle 12.

In the example shown in FIG. 5A, the allowable condition is not satisfied since the distance LM between the midpoints is shorter than 0.5 times the distance r₀. In the example of FIG. 5B, the allowable condition is satisfied since the distance LM between the midpoints is 0.5 times or more the distance r₀. In FIGS. 5A and 5B, only the monomer particles 21 and 22 are shown as a pair of monomer particles that are determination targets with respect to the position of the monomer particle 11 and the candidate position of the monomer particle 12, but the number of pairs of monomer particles that are determination targets is not limited to one.

In a case where it is determined in step SA09 of FIG. 4 that the distance LM between the midpoints does not satisfy the allowable condition, the current candidate position is canceled and a new candidate position is determined (steps SA05 and SA06). Ina case where the distance LM between the midpoints satisfies the allowable condition, a new monomer particle is disposed at the current candidate position (step SA10).

Then, it is determined whether or not the disposition of all the monomer particles in one polymer molecule model is completed (step SA11). In a case where the disposition is not completed, the processes of determining the candidate position of the monomer particle to be disposed next (steps SA05 to SA10) are repeated. In a case where the disposition of all the monomer particles in one polymer molecule model is completed, it is determined whether or not the disposition of all polymer molecule models is completed (step SA12) . In a case where the disposition of the polymer molecule model is not completed, the processes of disposing the new polymer molecule model (steps SA01 to SA11) are repeated. In a case where the disposition of all the polymer molecule models is completed, the process of disposing the polymer molecule models is terminated.

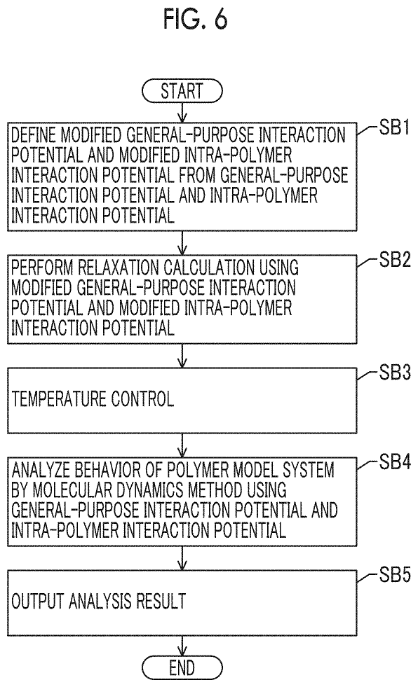

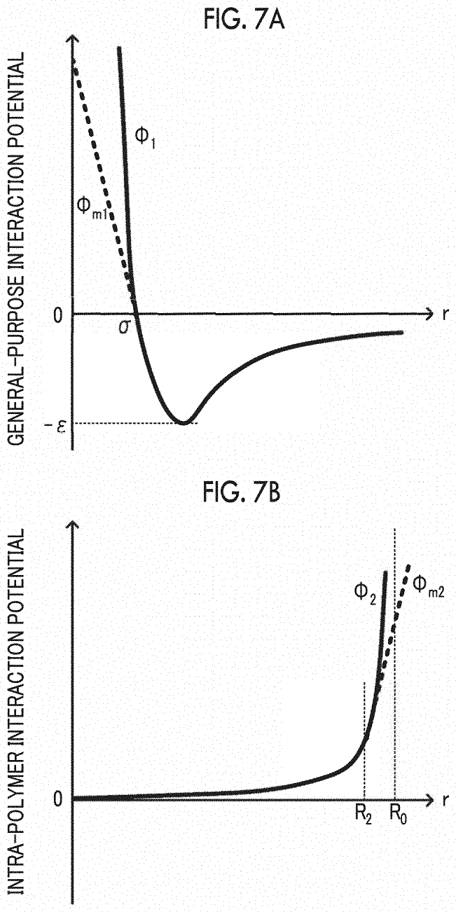

In a case where the disposition of the polymer molecule models is terminated, the relaxation calculation is performed, and then, a simulation of a behavior of the polymer molecule model is performed. Next, the relaxation calculation and the simulation will be described with reference to FIGS. 6 to 7B.

FIG. 6 is a flowchart of the relaxation calculation and the simulation. Each process shown in FIG. 6 is executed by the processing unit 51 (FIG. 2).

First, an interaction potential that is used in the relaxation calculation is defined on the basis of a general-purpose interaction potential and an intra-polymer interaction potential that is used in the simulation (step SB1).

For example, a Leonard Jones potential is used as the general-purpose interaction potential ϕ₁. In this case, the general-purpose interaction potential ϕ₁ is expressed by the following expression.

$$\begin{matrix}[ Equation 1 ] & \\ {φ}_{1} ( r ) = 4 ɛ ( {( \frac{σ}{r} )}^{12} - {( \frac{σ}{r} )}^{6} ) & ( 1 )\end{matrix}$$

Here, r represents a distance from a monomer particle, and ε and σ represent fitting parameters having a dimension of energy and a dimension of distance, respectively.

FIG. 7A is a graph showing a shape of the general-purpose interaction potential ϕ₁. ϕ₁(σ) is 0. The depth of the general-purpose interaction potential ϕ₁ is ε. In a range shorter than a distance at which the general-purpose interaction potential ϕ₁ has a minimum value, the general-purpose interaction potential ϕ₁ becomes large as the distance r approaches 0, and becomes infinite at r=0.

In the relaxation calculation, a modified general-purpose interaction potential ϕₘ₁ obtained by modifying the general-purpose interaction potential ϕ₁ is used as an interaction potential acting between all the monomer particles. The modified general-purpose interaction potential ϕₘ₁ has a shape in which an absolute value of an inclination is reduced within a range of the general-purpose interaction potential ϕ₁ that is equal to or less than a first distance.

In FIG. 7A, a portion of the modified general-purpose interaction potential ϕₘ₁ that is equal to or less than the first distance is indicated by a broken line. In the example shown in FIG. 7A, r=σ is adopted as the first distance. In the range of r≤σ, the modified general-purpose interaction potential ϕₘ₁ is linear, and its inclination is equal to an inclination of the general-purpose interaction potential ϕ₁ at r=σ.

The FENE potential is used as the intra-polymer interaction potential ϕ₂. In this case, the intra-polymer interaction potential ϕ₂ is expressed by the following expression.

$$\begin{matrix}[ Equation 2 ] & \\ {φ}_{2} ( r ) = - \frac{k}{2} {R}_{0}^{2} ln ( 1 - \frac{{r}^{2}}{{R}_{0}^{2}} ) & ( 2 )\end{matrix}$$

Here, k represents a spring constant of a spring that connects monomer particles, and R₀ represents a maximum extension distance of the spring.

FIG. 7B is a graph showing a shape of the intra-polymer interaction potential ϕ₂. ϕ₂ (0)=0, and ϕ₂ monotonically increases as the distance r increases. At r=R₀, ϕ₂ becomes infinite.

In the relaxation calculation, the modified intra-polymer interaction potential ϕₘ₂ obtained by modifying the intra-polymer interaction potential ϕ₂ is used as an interaction potential acting between two adjacent monomer particles in the polymer. The modified intra-polymer interaction potential ϕₘ₂ has a shape in which an absolute value of an inclination becomes small within a range of the intra-polymer interaction potential ϕ₂ that is equal to or greater than a second distance.

In FIG. 7B, a portion of the modified intra-polymer interaction potential ϕₘ₂ that is equal to or greater than the second distance is indicated by a broken line. In a range of r≥R₂, the modified intra-polymer interaction potential ϕₘ₂ is linear, and its inclination is equal to an inclination of the intra-polymer interaction potential ϕ₂ at r=R₂.

After defining the modified general-purpose interaction potential ϕₘ₁ and the modified intra-polymer interaction potential ϕₘ₂ used in the relaxation calculation, the relaxation calculation is performed by a molecular dynamics method using these interaction potentials (step SB2). In the relaxation calculation, a FIRE algorithm is applied to dissipate energy and to generate the most stable state of a system including a plurality of polymer molecule models.

Next, a specific application example of the FIRE algorithm will be described. In a case where an inner product of a force vector applied to a monomer particle and a velocity vector of the monomer particle is negative for each of the monomer particles during the relaxation calculation, the velocity of the monomer particle is set to zero. This means that in a case where the monomer particle has a velocity in a direction away from the most stable position, the velocity is set to zero to dissipate its kinetic energy. Thus, it is possible to settle the monomer particle at the most stable position in a short time.

The relaxation calculation is terminated at a time point when the energy of the system becomes lower than a certain reference value. For example, in a case where a temperature obtained from an average value of kinetic energies of all the monomer particles is equal to or lower than 0.1 K, the relaxation calculation is terminated. At the time point when the relaxation calculation is terminated, a state where all the monomer particles are almost stationary at the most stable positions is obtained.

Ina case where the relaxation calculation is terminated, a temperature control is performed (step SB3). In the temperature control, the general-purpose interaction potential ϕ₁ and the intra-polymer interaction potential ϕ₂ are used, and a calculation is performed by the molecular dynamics method using, for example, a velocity scaling method. At a time point when the temperature of the system including a plurality of polymer molecule models increases up to a target value, the temperature control is terminated. Thus, an equilibrium state in which the polymer molecule model is stably disposed in the initial disposition area is obtained.

Then, the behavior of the polymer molecule model is analyzed by executing a calculation by the molecular dynamics method using the general-purpose interaction potential ϕ₁ and the intra-polymer interaction potential ϕ₂ (step SB4). In this simulation, for example, the rotary vanes 61 (FIG. 3) are rotated. In a case where the analysis is terminated, the analysis result is output to the output unit 52 (FIG. 2).

Next, advantageous effects of the above embodiment will be described. At a location where the locked state shown in FIG. 1B occurs, midpoints of lines connecting monomer particles in a polymer molecule model are close to each other. In this embodiment, in the case of disposing monomer particles, as shown in FIG. 5A, in a case where the distance LM between the midpoints of the midpoint MPO of the line connecting the candidate position of the monomer particle 12 to be disposed next and the monomer particle 11 disposed immediately before and the midpoint MP1 of the line connecting two other monomer particles 21 and 22 is shorter than the distance r₃, the current candidate position of the monomer particle 12 is canceled and the candidate position is set again. Thus, it is possible to suppress the occurrence of the locked state as shown in FIG. 1B. Actually, in a case where a plurality of monomer particles were disposed by the method according to the present embodiment and relaxation calculation was performed, monomer particles having an extremely high potential energy as shown in FIG. 1C were not generated.

In a case where the monomer particles are disposed by the method according to the present embodiment, since the locked monomer particles are not generated, it is possible to perform a highly accurate simulation.

In a case where monomer particles having an extremely high potential energy are present (in a case where a polymer molecule model system is unstable), since a very large force is applied to the monomer particles, it is necessary to shorten a time step width so that the relaxation calculation does not fail. For example, in a simulation of a system that is in a stable state, a time step width determined from a sound velocity or a period of vibration is used, but in a case where the system is unstable, it is necessary to shorten a time step width compared with the above-mentioned time step. On the other hand, in the present embodiment, there is no monomer particle having an extremely high potential energy in a state where monomer particles are initially disposed. Thus, it is possible to set the time step width of the relaxation calculation to be substantially the same as a time step width in a case where an actual simulation is performed. As a result, it is possible to shorten a time necessary for the relaxation calculation.

Actually, the relaxation calculation was performed in a case where monomer particles were disposed by the related art voxel division method and a case where the monomer particles were disposed by the method according to the above-described embodiment. As a polymer system that is a simulation target, the number of monomer particles that form one polymer molecule model was 32, the number of polymer molecule models of a polymer system was 875, and the number of times of renormalization was 19. The fitting parameter ε of the general-purpose interaction potential ϕ₁ expressed by the equation (1) was set to 443K, and σ was set to 8.48×10⁻⁹ m. The spring constant k of the intra-polymer interaction potential ϕ₂ expressed by the equation (2) was set to 2.55×10⁻³ J/m², and the maximum extension distance R₀ was set to 1.27×10⁻⁸ m.

In a case where monomer particles are disposed by the related art voxel division method, in order to prevent the calculation from failing, the time step width of the relaxation calculation should be about 6×10⁻⁹ s. On the other hand, in a case where monomer particles are disposed by the method according to the present embodiment, the calculation does not fail even in a case where the time step width of the relaxation calculation was about 6×10⁻⁶ s. As a result, it is possible to shorten the time necessary for the relaxation calculation up to about 1/90.

In the related art method, trial and error is necessary to find an appropriate time step width where the calculation does not fail. Since the trial and error is necessary, it is difficult to automate the relaxation calculation. On the other hand, in a case where the method according to the above embodiment is applied, it is not necessary to perform the trial and error for finding the time step width of the relaxation calculation. Accordingly, it is possible to automate the relaxation calculation.

Further, in the above-described embodiment, during the relaxation calculation, the modified general-purpose interaction potential ϕₘ₁ and the modified intra-polymer interaction potential ϕₘ₂ with reduced inclinations are used in an area where the inclinations of the general-purpose interaction potential ϕ₁ and the intra-polymer interaction potential ϕ₂ are large. As a result, an excessively large force acting on a specific monomer particle is suppressed. Thus, an excellent effect that the failure of the relaxation calculation does not easily occur is obtained.

Further, in the above embodiment, since the FIRE algorithm is applied to the relaxation calculation, it is possible to shorten a time necessary for a system including a plurality of polymer molecule models to reach the most stable state.

The method according to the above embodiment for determining an initial disposition of monomer particles may be applied to a simulation of a particle system using a molecular dynamics method, a renormalization group molecular dynamics method, a particle dynamics method, or the like.

The above embodiment is exemplary, and the present invention is not limited to the above-described embodiment. For example, it will be apparent to those skilled in the art that various modifications, improvements, combinations, or the like may be made. It should be understood that the invention is not limited to the above-described embodiment, but may be modified into various forms on the basis of the spirit of the invention. Additionally, the modifications are included in the scope of the invention.

It should be understood that the invention is not limited to the above-described embodiment, but may be modified into various forms on the basis of the spirit of the invention. Additionally, the modifications are included in the scope of the invention.

What is claimed is:

1. A polymer molecule model disposition method for disposing a plurality of polymer molecule models each including a plurality of monomer particles in an initial disposition area in a virtual space that is a simulation target, comprising:
disposing one monomer particle in the initial disposition area;
determining a candidate of a position of a monomer particle to be disposed next as a candidate position; and
canceling, in a case where a distance between midpoints that is a distance from a midpoint of a line connecting a position of a monomer particle that is disposed immediately before to the candidate position of the monomer particle to be disposed next to a midpoint of a line connecting positions of two monomer particles included in a polymer molecule model that is already disposed does not satisfy an allowable condition, the candidate position that is determined immediately before, determining another position as a new candidate position, and disposing, in a case where the distance between midpoints satisfies the allowable condition, the monomer particle at the candidate position that is determined immediately before.

2. The polymer molecule model disposition method according to claim 1, further comprising:
determining the candidate position of the monomer particle to be disposed next so that the distance from the position of the monomer particle that is disposed immediately before to the candidate position of the monomer particle to be disposed next is equal to a distance between two consecutive monomer particles included in one polymer molecule model.

3. The polymer molecule model disposition method according to claim 1, further comprising:
randomly determining a direction from the position of the monomer particle that is disposed immediately before to the candidate position of the monomer particle to be disposed next.

4. The polymer molecule model disposition method according to claim 1, further comprising:
canceling, in a case where a distance between the candidate position that is determined immediately before and the position of the monomer particle that is already disposed does not satisfy an allowable condition of an inter-particle distance, the candidate position that is determined immediately before, and determining another position as a new candidate position.

5. The polymer molecule model disposition method according to claim 1, further comprising:
after the disposition of all the monomer molecule models is terminated and before simulating a behavior of a polymer molecule model using a general-purpose interaction potential acting between monomer particles and an intra-polymer interaction potential acting between adjacent monomer particles in the same polymer molecule model, performing relaxation calculation based on a molecular dynamics method using a modified general-purpose interaction potential in which an absolute value of an inclination is reduced in a range of the general-purpose interaction potential that is equal to or less than a first distance and a modified intra-polymer interaction potential in which an inclination is reduced in a range of the intra-polymer interaction potential that is equal to or greater than a second distance.

6. The polymer molecule model disposition method according to claim 5, further comprising:
performing a process of setting a velocity of a monomer particle of which an inner product of a velocity vector of the monomer particle and a force vector acting on the monomer particle is negative to zero, in the relaxation calculation.

7. The polymer molecule model disposition method according to claim 5, further comprising:
after performing the relaxation calculation, performing a temperature control of increasing a temperature of a system of the polymer molecule model up to a target value.

8. A simulation method comprising:
disposing a plurality of polymers in a virtual space that is a simulation target using the polymer molecule model disposition method according to claim 1; and
analyzing a behavior of a plurality of polymer molecule models using a molecular dynamics method.

9. A simulation apparatus comprising:
an input unit through which information for defining an initial disposition area of a polymer molecule model that is a simulation target is input; and
a processing unit that disposes a plurality of polymer molecule models each including a plurality of monomer particles in the initial disposition area defined by the information input through the input unit,
wherein the processing unit includes
a function of disposing one monomer particle in the initial disposition area,
a function of determining a candidate of a position of a monomer particle to be disposed next as a candidate position, and
a function of canceling, in a case where a distance between midpoints that is a distance from a midpoint of a line connecting a position of a monomer particle that is disposed immediately before to the position of the monomer particle to be disposed next to a midpoint of a line connecting positions of two consecutive monomer particles included in a polymer molecule model that is already disposed does not satisfy an allowable condition, the candidate position that is determined immediately before, and determining another position as a new candidate position.

10. A non-transitory computer-readable medium storing a program causing a computer to execute:
a function of acquiring information for defining an initial disposition area where a polymer molecule model that is a simulation target is disposed;
a function of disposing one monomer particle in the initial disposition area in disposing a plurality of polymer molecule models each including a plurality of monomer particles;
a function of determining a candidate of a position of a monomer particle to be disposed next as a candidate position; and
a function of canceling, in a case where a distance between midpoints that is a distance from a midpoint of a line connecting a position of a monomer particle that is disposed immediately before to the position of the monomer particle to be disposed next to a midpoint of a line connecting positions of two consecutive monomer particles included in a polymer molecule model that is already disposed does not satisfy an allowable condition of the distance between midpoints, the candidate position that is determined immediately before, and determining another position as a new candidate position.
